(19) United States
(12) Patent Application Publication
(10) Pub. No.: US 2022/0257691 A1
(43) Pub. Date: Aug. 18, 2022

(54) MEDICINE FOR TREATING PULMONARY MYCOSIS

(71) Applicant: Mingchun XIAO, Guangdong (CN)

(72) Inventor: Mingchun XIAO, Guangdong (CN)

(21) Appl. No.: 17/624,851

(22) PCT Filed: Aug. 23, 2019
(86) PCT No.: PCT/CN2019/102246

§ 371 Date: Jan. 5, 2022

(51) Int. Cl. A61K 36/8888; A61K 36/8988; A61K 36/8994; A61K 36/752; A61K 36/484; A61K 36/8966; A61K 36/428; A61K 36/31; A61K 36/483; A61K 36/904; A61K 36/185; A61K 36/28; A61K 36/898; A61K 36/714; A61K 36/72; A23L 33/00; A23L 33/105; A23K 10/30

(52) U.S. Cl. CPC A61K 36/8888; A23V 2002/00; A61K 36/8994; A61K 36/752; A61K 36/484; A61K 36/8966; A61K 36/428; A61K 36/31; A61K 36/483; A61K 36/904; A61K 36/185; A61K 36/28; A61K 36/898; A61K 36/714; A61K 36/72; A23L 33/40; A23L 33/105; A23K 10/30; A61K 2236/37; A61K 2236/331; A61K 2236/55; A61K 36/8988

(57) ABSTRACT

The application of the addition and subtraction of Er Chen Tang in the preparation of medicine or health care products for the treatment of pulmonary fungal diseases belongs to the medical and health care field, which specifically relates to the use of alkaloids and volatile oils contained in Pinellia ternate, Tangerine peel, Liquorice, Coix seed, Gastrodia elata in the preparation of medicine or health care products for the treatment of pulmonary fungal diseases.

### TECHNICAL FIELD

The invention belongs to the field of medical and health care, and relates to the new application of addition and subtraction of raw materials of Er Chen Tang in the preparation of drugs or health care products for the treatment of pulmonary fungal diseases, in particular to the new use of alkaloids and volatile oils contained in raw materials such as Pinellia ternata, Tangerine peel, Liquorice, coix seed, Gastrodia elata in the preparation of drugs or health care products for the treatment of pulmonary fungal diseases.

### BACKGROUND TECHNOLOGY

Modern medicine divides fungal diseases into superficial fungal diseases and deep fungal diseases. Superficial mycosis refers to the infection of keratin tissue (including cuticle, deck, hair, etc.). Deep mycosis, also known as invasive mycosis (IFD), refers to fungal infection caused by fungal invasion of human subcutaneous mucosa, blood and internal organs. The most common fungal pathogens are Candida, Aspergillus and others, such as Cryptococcus, Mucor, Pneumocystis, etc. According to the incomplete statistics of GAFFI, 300 million people suffer from severe fungal infections every year, and more than 25 million people have a very high risk of death or blindness. The mortality of IFD was 30% to 40% for Candida and 40% to 90% for Aspergillus. According to statistics, there are more than 8 million cases of invasive Aspergillus infection and Candida infection in China. Recent epidemiological survey data show that there are 100 million patients with chronic obstructive pulmonary disease (COPD), including more than 10 million patients with chronic pulmonary aspergillosis (CPA) and allergic bronchopulmonary aspergillosis (ABPA). But at present, the research on the diseases caused by fungal infection is very limited, and the drugs on the market for the treatment of deep fungal infection often have large side effects, long-term use can cause damage to the body organs, and the curative effect is not ideal, at the same time, produce many adverse reactions. However, the patent “cracking the pathogenic factors of cancer and traditional Chinese medicine treatment method” (application No.: 201310274489.7) and the international patent “a pharmaceutical composition for the treatment of cancer and its preparation method” (application No.: PCT/CN2013/080936, entry into China application No.: 201380075486.9, entry into the U.S. Pat. No. 9,498,508B2) previously applied by the applicant involve the traditional Chinese medicine treatment method for fungal infection Among them, “Er Chen Tang” is the basic prescription for the treatment of fungal infection. Another patent application of the applicant, “a drug for the treatment of chronic pulmonary aspergillosis” (application No.: 201910499923.9), relates to the treatment of chronic pulmonary aspergillosis with “Pinellia, Atractylodes Macrocephala and Gastrodia Tang”. Therefore, this application should be continued on this basis.

In recent years, medical research has proved that the main transmission routes of Aspergillus, cryptococcosis, mucormycosis and other fungi are respiratory tract and skin, which can lead to systemic cryptococcosis, pulmonary aspergillosis, mucormycosis rhinocerebri, pulmonary mucormycosis, gastrointestinal mucormycosis, disseminated mucormycosis and so on.

Modern medical research has also proved that the main way of infection of aspergillosis is to inhale spores in the air. Aspergillus spores enter the sinuses and lungs with breathing, germinate and produce hyphae, and enter the cells for qualitative change. Aspergillus prefers to invade blood vessels.

Candida albicans can cause infection of skin, mucous membrane, viscera, bone and brain through exogenous or endogenous ways. Visceral infection mainly includes pneumonia, bronchitis, esophagitis, enteritis and nephritis.

Cryptococcus neoformans is mainly inhaled through the respiratory tract. The first site of infection may be the lung. From the lung, it can spread to other parts of the body, including the skin, bone, heart, etc., and the most vulnerable is the central nervous system, causing chronic meningitis.

Chronic pulmonary aspergillosis refers to chronic pulmonary aspergillosis. Aspergillus is widely distributed in nature. It exists in air, food, soil, clothes and furniture. It can also be isolated from the skin and mucous membrane of normal people. Aspergillus is also a conditional pathogen. When the immune function is inhibited or damaged due to tissue damage or chronic diseases, it is easy to cause disease. Pulmonary aspergillosis is mostly exogenous infection. The main route of infection is inhalation of Aspergillus spores in respiratory tract to cause pulmonary aspergillosis, or invasion of blood flow to spread to all organs of the body. According to medical mycology edited by Wen Hai, pulmonary aspergillosis can be divided into four types:

Type 1, pulmonary Aspergillus ball: generally no obvious systemic symptoms, but can have irritating cough and repeated massive hemoptysis.

Type 2, allergic aspergillosis: chills, fever, fatigue, irritating cough, with obvious asthma. Cough brown yellow purulent sputum, sometimes with blood. Dry and wet rales could be heard in the lesion.

Type 3, bronchial pneumonia aspergillosis: cough, expectoration, low fever, fatigue, loss of appetite, etc. When the lesion invaded the lung parenchyma, it showed pneumonia, necrosis and purulence, forming lung abscess. There may be signs of pulmonary parenchymal lesions and moist rales. Imaging showed pulmonary cavity, destruction or fibrosis of lung parenchyma around cavity, obvious pleural thickening, and Aspergillus empyema.

Type 4, secondary pulmonary aspergillosis: primary disease, fever, cough, expectoration, hemoptysis, chest pain, dyspnea, etc. There may be dry and wet rales in both lungs.

The main symptoms are low fever, cough of white sticky sputum or colloidal massive sputum, sometimes blood can be seen in the sputum. Bronchogenic, pneumonia or tuberculous symptoms, lung moist rales, pleurisy or pleural effusion may occur.

“Er Chen Tang” is an expectorant, and its effect is to dry and damp phlegm. The raw materials of Er Chen Tang are composed of pinellia, orange peel, Poria and licorice. “Pinellia, Atractylodes Macrocephala and Gastrodia Tang” is a flavor added to “Er Chen Tang”. On the basis of the original dry and wet phlegm, the herbal medicine of invigorating spleen and Gastrodia elata, which is composed of pinellia, Gastrodia elata, Poria, orange peel, Atractylodes and licorice, is added. The addition and reduction of Er Chen Tang can be used to treat wet phlegm, hot phlegm, cold phlegm and other phlegm diseases, and is often used in patients with chronic bronchitis and chronic gastritis. However, there has been no literature report on the treatment of pulmonary mycosis in Er Chen Tang.

Pinellia ternata is a kind of medicine for warming and resolving cold phlegm, which can return to spleen, stomach and lung channels. Efficacy: dry dampness and remove phlegm, reduce adverse reactions and stop vomiting, eliminate swelling and relieve pain for external use, and treat dampness phlegm and cold phlegm syndrome. Modern studies have shown that Pinellia ternate contains volatile oils and alkaloids, which have anti-tumor and antitussive effects. According to the research results of Zeng Jianhong (Department of biology, West China Normal University, nanchong-2015), it was found that the total alkaloid content of Pinellia ternata in different growth stages was significantly different, and the average total alkaloid yield of each plant was the highest in the concentrated seedling stage. The test results showed that the sample weight was 0.739 g, the total alkaloid content was 0.099%, the average dry weight of a single plant was 1.858 g, and the average total alkaloid yield of a single plant was 0.1839 g. The ethanol extract of Pinellia ternate has broad-spectrum antibacterial activity, and also has a certain effect on the inhibition of fungi. However, it has not been reported that Pinellia alkaloids and volatile oil have the effect of anti pulmonary fungal disease.

Tangerine peel is a kind of medicine for regulating qi, which is attached to Pericarpium Citri Reticulatae and can return to spleen and lung meridian. Modern research shows that fresh Tangerine Peel Decoction has the effect of dilating trachea, volatile oil has the effect of stimulating expectorant, and water-soluble total alkaloids of Pericarpium Citri Reticulatae have the effect of increasing hypertension. The contents of volatile oil and alkaloids in Pericarpium Citri Reticulatae were 1.9%-3.5% and 0.240%-0.636%, respectively. However, the alkaloids and volatile oil of tangerine peel have not been used in the treatment of pulmonary mycosis.

Liquorice is a kind of Tonifying Qi medicine, which can return to heart, lung, spleen and stomach channels. Contains alkaloids, polysaccharides and other components, with cough expectorant effect. According to the report of Li Jie et al. (China food additive-2018) on the optimization of extraction process of alkaloids, the optimal extraction process parameters of Liquorice alkaloids were as follows: ethanol concentration 87%, extraction time 90 min, extraction temperature 53° C., solid-liquid ratio 1:20 (g/ml). Under these conditions, the yield of Liquorice alkaloids could reach 0.3659 mg/g However, there have been no reports on the use of alkaloids and volatile oil of Liquorice in the treatment of pulmonary mycosis.

Coix seed is a diuretic and detumescence medicine, which can return to spleen, stomach and lung meridian. Coix seed decoction, alcohol and acetone extracts have obvious inhibitory effect on cancer cells. Its fatty oil can reduce serum calcium and blood sugar, and has antipyretic, sedative and analgesic effects. However, there have been no reports about the use of Coix seed alkaloids and volatile oil in the treatment of pulmonary mycosis

Gastrodia elata is a medicine for relieving wind and spasm, which belongs to the liver meridian. It is a medicine for headache, numbness, rheumatism and arthralgia. According to the research of Guan Ping et al. [Journal of Southwest Normal University (NATURAL SCIENCE EDITION), 2008], it is found that the volatile oil of Gastrodia elata has obvious inhibitory effect on Aspergillus oryzae and Aspergillus flavus in agricultural products, and the volatile oil of Gastrodia elata has significant antitussive and good expectorant effect. However, there have been no reports about the use of alkaloids and volatile oil of Gastrodia elata in the treatment of pulmonary mycosis.

The basis of natural medicine treatment and prevention lies in its active ingredients. The chemical composition of any natural medicine is very complex. Alkaloid is one of the important active components in plant medicine. There are many kinds of alkaloids known. Most of them are alkaline and most of them are crystalline. Alkaloids are all nitrogen-containing organic compounds, most of them have complex ring structure, and nitrogen is mostly contained in the ring. The alkalinity is related to the structure type of alkaloids. Many scholars have found that alkaloids have many pharmacological activities, including anti-tumor, anti-virus, anti-inflammatory, antibacterial, analgesic, liver protection, cardiovascular and many other aspects. For example, morphine obtained from opium has strong analgesic effect; codeine has cough relieving effect, papaverine has smooth muscle relaxing effect; ephedrine in Ephedra has antiasthmatic effect; berberine in Coptis chinensis and Phellodendron amurense has antibacterial effect; scopolamine in Datura, tianxianzi and belladonna has antispasmodic effect, etc. These alkaloids are the most studied at present, and their effects are different. However, the pharmacological mechanisms of alkaloids in anti-tumor, anti-virus, anti-inflammatory, antibacterial, analgesic, liver protection and cardiovascular effects have not been fully studied. There have been no reports about the effect of alkaloids in the treatment of pulmonary mycosis.

The extraction method of alkaloids can be determined by organic solvent according to the characteristics that alkaloids can be dissolved in water and alkaloids are not soluble in water. Alkaloids are mostly soluble in methanol, ethanol, ether, chloroform and other organic solvents, which are generally insoluble or difficult to dissolve in water. Based on this characteristic, alkaloids can be extracted from plants with different solvents. The common methods are cold soak, percolation, ultrasonic, Soxhlet extraction, hot reflux extraction and so on. The new technology of alkaloid extraction, 1. Membrane separation method, has the advantages of high separation efficiency and mild conditions without adding chemical reagents. 2. Supercritical fluid extraction has high efficiency, high purity, mild conditions, no pollution, and can change temperature and pressure.

Volatile oil is the general name of a kind of oil-like liquid which has aromatic smell and can volatilize at room temperature, and it is widely distributed in the plant kingdom. Volatile oil is mainly composed of terpenoids, aromatic compounds and their oxygen-containing derivatives, such as alcohols, aldehydes, ketones, phenols, ethers, endolipids, etc., in addition to nitrogen-containing and sulfur-containing compounds. Modern pharmacological experiments show that volatile oil has anti-inflammatory, anti allergic, anti microbial, anti mutation and anti-cancer, anthelmintic effect, enzyme inhibition, effect on central nervous system, effect on respiratory system, etc. However, the understanding and Research on the active mechanism or pharmacological mechanism of volatile oil is still very limited. It has not been reported that volatile oil can treat pulmonary mycosis.

The extraction method of volatile oil can choose different extraction methods, such as 1. Steam distillation: the extraction is carried out by using the properties of volatile oil and water immiscibility. 2. Organic solvent extraction method: petroleum ether, ether and other organic solvents are used for continuous reflux extraction or cold immersion extraction. 3. In supercritical fluid extraction, the volatile oil was extracted by supercritical fluid of carbon dioxide. 4. The cold pressing method is suitable for the extraction of fresh plants with high oil content.

### SUMMARY OF THE INVENTION

The invention aims to provide a medicine for treating pulmonary mycosis, in particular to provide a new use of alkaloids and volatile oils contained in plant medicine Pinellia ternate, Tangerine peel, Liquorice, Coix seed and Gastrodia elata in the preparation of medicine or health care product for treating pulmonary fungal diseases.

Furthermore, the medicine for treating pulmonary mycosis provided by the invention is characterized in that the raw material of the medicine is composed of alkaloids and/or volatile oils extracted from Pinellia ternata, alkaloids and/or volatile oils extracted from tangerine peel, alkaloids and/or volatile oils extracted from Liquorice, alkaloids and/or volatile oils extracted from Coix seed, alkaloids and/or volatile oils extracted from Gastrodia elata composition.

The preparation method of the drug is as follows: the alkaloids of Pinellia ternata, the alkaloids of Tangerine peel, the alkaloids of Coix seed, the alkaloids of Liquorice and the alkaloids of Gastrodia elata are mixed in a certain proportion, and various conventional excipients needed for the preparation of different formulations are added to prepare the pharmaceutically acceptable formulations.

The preparation method of the medicine is as follows: the volatile oils of Pinellia ternata, the volatile oils of fresh orange peel, the volatile oils of Liquorice, the volatile oils of Coix seed and the volatile oils of Gastrodia elata are mixed in a certain proportion, and various conventional auxiliary materials needed for the preparation of different formulations are added to prepare the pharmaceutical acceptable formulations.

The preparation method of the drug is as follows: take the alkaloids and volatile oils of Pinellia ternata, Tangerine peel alkaloids and volatile oils, Liquorice alkaloids and volatile oils, Coix seed alkaloids and volatile oils, Gastrodia elata alkaloids and volatile oils, mix various volatile oils in proportion, then mix them with various alkaloids dry and proper auxiliary materials after inclusion with betachyclodextrin, and then make tablets by conventional preparation methods The pharmaceutically acceptable dosage forms such as granules.

Further, the preparation method and the extraction method of alkaloids can be determined by using the modern commonly used organic solvent, and alkaloids can be extracted from Pinellia ternata, Tangerine peel, Liquorice, Coix seed and Gastrodia elata with different solvents. The supercritical fluid extraction method is preferred, followed by membrane separation or ultrasonic extraction or Soxhlet extraction.

The extraction methods of volatile oils from Pinellia ternata, Tangerine peel, Liquorice, Coix seed and Gastrodia elata were used in modern common methods. The supercritical fluid extraction method or water vapor distillation method is selected for Pinellia terna, Liquorice, Coix seed and Gastrodia elata, and then the organic solvent extraction method is selected: the continuous reflux extraction or cold leaching method is used for the organic solvents such as petroleum ether and ether; the cold pressure method or water vapor distillation method is preferred for the extraction of fresh orange peel.

Furthermore, the medicine provided by the invention is characterized in that the raw materials of the medicine can be selectively increased according to clinical symptoms: alkaloids and/or volatile oils extracted from Fritillaria cirrhosa, alkaloids and/or volatile oils extracted from Trichosanthes kirilowii, alkaloids and/or volatile oils extracted from Rhizoma arisaematis, alkaloids and/or volatile oils extracted from Sinapis alba, alkaloids and/or volatile oils extracted from Gleditsia sinensis, alkaloids and/or volatile oils extracted from Radix Stemona, alkaloids and/or volatile oils extracted from Japanese ardisia, alkaloids and/or volatile oils extracted from Artichoke, alkaloids and/or volatile oils extracted from Bletilla striata, alkaloids and/or volatile oils extracted from Lotus root, alkaloids and/or volatile oils extracted from Aconitum carmichaeli, alkaloids and/or volatile oils extracted from Hovenia dulcis.

Furthermore, the medicine provided by the invention is characterized in that the weight parts of the alkaloids and/or volatile oils extracted from Pinellia ternata are 0.001-50 parts by weight, the alkaloids and/or volatile oils extracted from Tangerine peel are 0.001-50 parts by weight, the alkaloids and/or volatile oils extracted from Coix seed are 0.001-50 parts by weight, the alkaloids and/or volatile oils extracted from Liquorice are 0.001-50 parts by weight, the alkaloids and/or volatile oils extracted from Gastrodia elata are 0.001-50 parts by weight, the alkaloids and/or volatile oils extracted from Fritillaria cirrhosa are 0-50 parts by weight, the alkaloids and/or volatile oils extracted from Trichosanthes kirilowii are 0-50 parts by weight, the alkaloids and/or volatile oils extracted from Rhizoma arisaematis are 0-50 parts by weight, the alkaloids and/or volatile oils extracted from Sinapis seeds are 0-50 parts by weight, The alkaloids and/or volatile oils extracted from Gleditsia sinensis are 0-50 parts by weight, The alkaloids and/or volatile oils extracted from Stemona are 0-50 parts by weight, The alkaloids and/or volatile oils extracted from Japanese ardisia are 0-50 parts by weight, The alkaloids and/or volatile oils extracted from Artichoke are 0-50 parts by weight, The alkaloids and/or volatile oils extracted from Bletilla striata are 0-50 parts by weight, The alkaloids and/or volatile oils extracted from Lotus root are 0-50 parts by weight, the alkaloids and/or volatile oils extracted from Aconitum carmichaeli are 0-50 parts by weight, the alkaloids and/or volatile oils extracted from Hovenia dulcis are 0-50 parts by weight.

Furthermore, the medicine is characterized in that the preparation method of the medicine is as follows: extracting alkaloids from each raw material medicine respectively, mixing various alkaloids in proportion, adding various conventional excipients required for preparing different dosage forms, and making pharmaceutically acceptable dosage forms such as tablets, powder injections, capsules, etc. by the conventional method of drug preparation.

Furthermore, the medicine is characterized in that the preparation method of the medicine is as follows: extracting volatile oils from each raw material medicine, mixing various volatile oils in proportion, adding various conventional auxiliary materials required for preparing different dosage forms, and making pharmaceutically acceptable dosage forms such as tablets, soft capsules, topical liniment, granules, etc. by the conventional method of drug preparation.

Furthermore, the medicine is characterized in that the preparation method of the medicine is as follows: extracting alkaloids and volatile oils from each raw material medicine respectively, mixing various volatile oils in proportion, and then mixing them with various alkaloids dry matter and appropriate amount of excipients after inclusion by beta cyclodextrin, and making pharmaceutically acceptable dosage forms such as tablets, granules, etc. by the conventional preparation method of medicine.

The application scope of the medicine or health care product for treating pulmonary mycosis includes the application in the fields of medical medicine, health food, health care products, animal medicine and animal feed.

The pharmacology of various raw materials of the medicine of the invention is as follows:

Pinellia ternate is the medicine for warming and resolving cold phlegm. The medicine is pungent, warm and can return to spleen, stomach and lung channels. Efficacy: dry dampness and remove phlegm, reduce adverse reactions and stop vomiting, eliminate swelling and relieve pain for external use, and treat dampness phlegm and cold phlegm syndrome. This shows that Pinellia ternate can treat “dampness evil” and “cold evil”. Modern research on Pinellia ternate contains volatile oil and alkaloids. The ethanol extract of Pinellia ternate has broad-spectrum antibacterial activity, and also has a certain effect on the inhibition of fungi. In the technical scheme provided by the invention, Pinellia ternate is the sovereign medicine, because the main pathological change of pulmonary mycosis is pulmonary Aspergillus or Candida and other fungal infection, all kinds of fungi will cause pulmonary inflammation and edema, resulting in a large amount of sputum accumulated in the lung, forming wet sputum, which is difficult to cough up, Pinellia ternate is the first important medicine for resolving phlegm and relieving cough, when Aspergillus infection, Pinellia ternate combined with Trichosanthes kirilowii and Fritillaria thunbergii can treat phlegm heat and chest. When Candida infection, Pinellia ternata combined with Arisaema, Sinapis alba, Gleditsia to enhance the effect of phlegm. The main effective components of Pinellia ternate against various fungi are alkaloids and volatile oil. According to the research results of Zeng Jianhong (Department of biology, West China Normal University, Nanchong), etc., it was found that the total alkaloid content of Pinellia ternata in different growth stages was significantly different, and the average total alkaloid yield of each plant was the highest in the concentrated seedling stage. The test results showed that the sample weight was 0.739 g, the total alkaloid content was 0.099%, the average dry weight of a single plant was 1.858 g, and the average total alkaloid yield of a single plant was 0.1839 g.

Tangerine Peel is a kind of medicine for regulating qi, which is attached to Pericarpium Citri Reticulatae and can return to spleen and lung meridian. Modern research shows that fresh Tangerine Peel Decoction has the effect of dilating trachea, volatile oil has the effect of stimulating expectorant, and water-soluble total alkaloids of Pericarpium Citri Reticulatae have the effect of increasing hypertension. The main effective components of orange red against various fungi are alkaloids and volatile oils.

Coix seed is a diuretic and detumescence medicine, which can return to spleen, stomach and lung meridian. The invention replaces Poria with coix seed. Because of the heart, spleen and kidney meridian of Poria cocos, the main chemical composition is polysaccharide, while coix seed is lung meridian, which can treat chest pain of lung carbuncle and purulent phlegm. The main effective components of Coix seed against various fungi are alkaloids and volatile oils.

Liquorice is a tonic medicine, Liquorice returns to heart, lung, spleen and stomach meridian. It has the function of relieving cough and removing phlegm, and also has the function of adjusting the drug resistance. Traditional Chinese medicine believes that “spleen deficiency creates phlegm dampness”, that is to say, fungi invade liver blood vessels and cause disseminated blood infection. After pulmonary infection, pulmonary inflammation is formed, and a large amount of sputum is produced to block the respiratory tract, which causes difficulty of oxygen and carbon dioxide exchange. “Qi Deficiency” is hypoxia, and the use of Liquorice and Qi regulating medicine Tangerine Peel can promote the exchange of oxygen and carbon dioxide. The main chemical components of Glycyrrhiza against various fungi are alkaloids, volatile oils and polysaccharides.

Gastrodia elata is a medicine for relieving wind and stopping spasm, which belongs to the liver meridian. Modern studies have found that the volatile oil of Gastrodia elata has significant antitussive and good expectorant effects. Due to the spread of fungal blood infection, liver meridian is the most vulnerable vein, and the brain is rich in blood vessels, so the brain is also a place where all kinds of fungi are easy to stay. Due to the blood-brain barrier, many drugs can not enter the brain, and Gastrodia elata is an antifungal drug targeting on the brain, so Gastrodia elata can assist Pinellia ternata to remove the fungal infection of upper respiratory tract. The main effective components of Gastrodia elata against various fungi are alkaloids and volatile oils.

Trichosanthes kirilowii and Fritillaria thunbergii belong to the lung, stomach and large intestine meridians, while Fritillaria thunbergii belongs to the lung and heart meridians. Trichosanthes kirilowii and Fritillaria thunbergii are important drugs for the treatment of lung dryness and heat, yellow phlegm obstructing the lung and sticky sputum. Because Aspergillus is Aspergillus flavus, its sputum is yellow pus sputum. In modern research, Fritillaria thunbergii contains a variety of alkaloids, which have antitussive effect. The main antifungal constituents of Trichosanthes kirilowii and Fritillaria thunbergii are alkaloids and volatile oils.

Rhizoma Arisaema, semen Sinapis alba, Gleditsia sinensis and Pinellia ternata are the same drugs for warming and resolving cold phlegm. Rhizoma Arisaema belongs to lung, liver and spleen meridians, Sinapis alba to lung, stomach meridians, Gleditsia sinensis to lung, large intestine meridians. Rhizoma Arisaema decoction has expectorant, anticonvulsant, sedative and analgesic effects. Modern research shows that Rhizoma Arisaema alkaloids can resist the toxicity of aconitine. Sinapis alba oil has a strong stimulating effect, can cause skin congestion, so there is no need for pulmonary hemorrhage. Gleditsia sinensis is used in the treatment of chronic pulmonary obstruction. It has strong phlegm resolving power, but it has a strong stimulating effect on gastric mucosa. According to different symptoms, Rhizoma Arisaema, semen Sinapis alba and Gleditsia sinensis can be selected to assist Pinellia ternata to enhance the effect of resolving phlegm and relieving cough. The main antifungal constituents of Arisaema, Sinapis alba and Gleditsia sinensis are alkaloids and volatile oils.

Radix Stemonae and Japanese ardisia are cough and asthma drugs, Radix Stemonae are lung meridians, Japanese ardisia is lung and liver meridians. Stemonae and Japanese ardisia have the function of moistening lung and relieving cough. Because of fungal infection, the lung is blocked by pus and phlegm, which leads to hypoxia, which is prone to asthma. In modern research, hundreds of alkaloids are contained, which can reduce the excitability of respiratory center and inhibit cough reflex. In vitro test has inhibition effect on many kinds of bacteria and cocci, and also on skin fungi. Dwarf tea has a significant cough and phlegm removing effect, but also has the effect of eliminating edema. The effective components of anti fungi of Stemonae and Japanese ardisia are alkaloids and volatile oils.

Artichoke, Bletilla striata and Lotus root are hemostatic drugs, while Artichokes are blood cooling hemostatics, and the heart is the heart and liver meridian. Bletilla striata and Lotus root joint are astringent hemostatic drugs, and belong to lung, stomach and liver channels. When the hemoptysis or hemoptysis is serious, it can increase the hemostasis of thistle, Bletilla striata and Lotus root. Alkaloids and volatile oils are the main effective components of artichoke, Bletilla striata and Lotus root.

Aconitum carmichaeli is a kind of medicine for warming internal organs, which can return to the heart, kidney and spleen meridians. It can quickly restore renal function, diuresis and eliminate edema. Aconitine in Aconitum carmichaeli belongs to diterpenoid alkaloids. The toxicity of aconitine formed by hydrolysis can be significantly reduced. Hovenia dulcis Thunb Is a diuretic and detumescence medicine, which can return to spleen meridian. Aconite and Hovenia dulcis can assist Pinellia ternate and Coix seed to eliminate the inflammatory edema caused by fungi. The main antifungal components of Aconitum carmichaeli and Hovenia dulcis are alkaloids and volatile oils.

### SPECIFIC IMPLEMENTATION MODE

### Example 1

The composition of the medicine for the treatment of pulmonary mycosis in the embodiment is as follows: alkaloids and volatile oils contained in 80 g fresh Pinellia ternata, alkaloids and volatile oils contained in 50 g fresh Tangerine peel, alkaloids and volatile oils contained in 100 g fresh Coix seed, alkaloids and volatile oils contained in 50 g fresh Liquorice, alkaloids and volatile oils contained in 80 g fresh Gastrodia elata.

According to the weight of selected raw materials, the alkaloids of Pinellia ternate, Tangerine peel, Liquorice, Coix seed and Gastrodia elata were extracted by supercritical fluid extraction or membrane separation method respectively. The alkaloids were mixed and added with conventional excipients. The tablets or powder injections and other pharmaceutically acceptable dosage forms were prepared according to the conventional preparation process.

According to the weight of selected raw materials, the volatile oils were extracted from Pinellia, Tangerine peel, Liquorice, Coix seed and Gastrodia elata by supercritical fluid extraction, organic solvent extraction or steam distillation, respectively. The volatile oils were mixed and added with conventional excipients to prepare soft capsules or granules and other pharmaceutically acceptable dosage forms.

According to the weight of the selected raw materials, the alkaloids and volatile oils were extracted from Pinellia ternate, Tangerine peel, Liquorice, Coix seed and Gastrodia elata by supercritical fluid extraction, organic solvent extraction or steam distillation, respectively. The volatile oils were mixed in proportion and then mixed with various alkaloids and appropriate excipients after inclusion by beta cyclodextrin It can be made into tablets, granules and other pharmaceutically acceptable dosage forms.

### Example 2

The composition of the medicine for treating pulmonary aspergillosis in the embodiment is as follows: alkaloids and volatile oils in 60 g fresh Pinellia ternata, alkaloids and volatile oils in 100 g fresh Gastrodia elata, alkaloids and volatile oils in 40 g fresh Tangerine peel, alkaloids and volatile oils in 40 g fresh Liquorice, alkaloids and volatile oils in 120 g fresh Coix seed, alkaloids and volatile oils in 60 g fresh Stemona, alkaloids and volatile oils in 100 g fresh Trichosanthes kirilowii, alkaloids and volatile oils in 50 g fresh Fritillaria cirrhosa, alkaloids and volatile oils in 80 g fresh Bletilla striata.

According to the weight of selected raw materials, the alkaloids of Pinellia ternate, Tangerine peel, Liquorice, Coix seed, Gastrodia elata, Stemona, Trichosanthes kirilowii, Fritillaria cirrhosa and Bletilla striata were extracted by supercritical fluid extraction or membrane separation method respectively. The alkaloids were mixed and added with conventional excipients, and the pharmaceutically acceptable dosage forms such as tablets or powder injections were made according to the normal preparation process.

According to the weight of selected raw materials, the volatile oils were extracted from Pinellia ternata, Tangerine peel, Liquorice, Coix seed, Gastrodia elata, Stemona, Trichosanthes kirilowii, Fritillaria cirrhosa and Bletilla striata by supercritical fluid extraction, organic solvent extraction or steam distillation, respectively. Then the volatile oils were mixed with conventional excipients and made into soft capsules or granules according to the conventional preparation process Acceptable dosage form.

According to the weight of the selected raw materials, the alkaloids and volatile oils were extracted from Pinellia ternata, Tangerine, Coix seed, Liquorice, Gastrodia elata, Stemona, Trichosanthes kirilowii, Fritillaria cirrhosa and Bletilla striata respectively by supercritical fluid extraction, organic solvent extraction or steam distillation. The volatile oils were mixed in proportion and then mixed with various alkaloids and appropriate excipients after inclusion by beta cyclodextrin The tablets, granules and other pharmaceutically acceptable dosage forms were prepared by conventional method.

### Example 3

The composition of the medicine for treating pulmonary candidiasis in the embodiment is as follows: alkaloids and volatile oils in 100 g fresh Pinellia ternata, alkaloids and volatile oils in 80 g fresh Tangerine peel, alkaloids and volatile oils in 50 g fresh Liquorice, alkaloids and volatile oils in 60 g fresh Coix seed, alkaloids and volatile oils in 100 g fresh Gastrodia elata, alkaloids and volatile oils in 60 g fresh Rhizoma Arisaematis, alkaloids and volatile oils in 100 g fresh Sinapis alba.

According to the weight of the selected raw materials, the alkaloids of Pinellia ternate, Tangerine peel, Coix seed, Liquorice, Gastrodia elata, Rhizoma Arisaematis and Sinapis alba were extracted by supercritical fluid extraction or membrane separation method respectively. The alkaloids were mixed and added into the conventional excipients, and the pharmaceutically acceptable dosage forms such as tablets or powder injections were made according to the conventional preparation process.

According to the weight of the selected raw materials, the volatile oils were extracted from Pinellia ternata, Tangerine peel, Coix seed, Liquorice, Gastrodia elata, Arisaema and Sinapis alba by supercritical fluid extraction, organic solvent or steam distillation, respectively. Then the volatile oils were mixed and added with conventional excipients to prepare soft capsules or granules Type.

According to the weight of the selected raw materials, the alkaloids and volatile oils were extracted from Pinellia ternate, Tangerine peel, Coix seed, Liquorice, Gastrodia elata, Arisaema and Sinapis alba by supercritical fluid extraction, organic solvent extraction or steam distillation, respectively. The volatile oils were mixed in proportion, then were inclusion by beta cyclodextrin, and then mixed with various alkaloids dry matter and appropriate accessories The preparation method of the drug is to prepare tablets, granules and other pharmaceutically acceptable dosage forms.

### Example 4

The composition of the medicine for treating pulmonary mycosis in case of blood in sputum or hemoptysis in the embodiment is as follows: alkaloids and volatile oils in 70 g fresh Pinellia ternata, alkaloids and volatile oils in 50 g fresh Tangerine peel, alkaloids and volatile oils in 50 g fresh Liquorice, alkaloids and volatile oils in 150 g fresh Coix seed, alkaloids and volatile oils in 100 g fresh Gastrodia elata, alkaloids and volatile oils in 120 g fresh Trichosanthes kirilowii, alkaloids and volatile oils in 150 g fresh Lotus root, alkaloids and volatile oils in 100 g fresh Artichoke, alkaloids and volatile oils in 100 g fresh Bletilla striata.

According to the weight of the selected raw materials, the alkaloids were extracted from Pinellia ternate, Tangerine peel, Coix seed, Liquorice, Gastrodia elata, Trichosanthes kirilowii, Lotus root, Artichoke and Bletillae striata respectively by supercritical fluid extraction or membrane separation method. The alkaloids were mixed and added with conventional excipients to prepare tablets or powder injections and other pharmaceutically acceptable dosage forms.

According to the weight of selected raw materials, the volatile oils were extracted from Pinellia ternata, Tangerine peel, Coix seed, Liquorice, Gastrodia elata, Trichosanthes kirilowii, Lotus root, Artichoke and Bletilla striata by supercritical fluid extraction, organic solvent extraction or steam distillation, respectively. Then the volatile oils were mixed with conventional excipients and made into soft capsules or granules according to the conventional preparation process The dosage form affected.

According to the weight of the selected raw materials, the alkaloids and volatile oils were extracted from Pinellia ternate, Tangerine peel, Coix seed, Liquorice, Gastrodia elata, Trichosanthes kirilowii, Lotus root, Artichoke and Bletilla striata by supercritical fluid extraction, organic solvent extraction or steam distillation, respectively The tablets, granules and other pharmaceutically acceptable dosage forms were prepared by the conventional method.

### Example 5

The composition of the medicine for treating pulmonary cryptococcosis in the embodiment is as follows: alkaloids and volatile oils in 100 g of fresh Pinellia ternata, alkaloids and volatile oils in 50 g of fresh Tangerine peel, alkaloids and volatile oils in 50 g of fresh Liquorice, alkaloids and volatile oils in 120 g of fresh Coix seed, alkaloids and volatile oils in 100 g of fresh Gastrodia elata, alkaloids and volatile oils in 100 g of fresh Rhizoma Arisaematis, alkaloids and volatile oils in 150 g fresh Trichosanthes kirilowii.

According to the weight of the selected raw materials, the alkaloids were extracted from Pinellia ternate, Tangerine peel, Coix seed, Liquorice, Gastrodia elata, Rhizoma Arisaematis and Trichosanthes kirilowii respectively by supercritical fluid extraction or membrane separation method. The alkaloids were mixed, added with conventional auxiliary materials, and the pharmaceutical acceptable formulations such as tablet or powder injection were made according to the conventional preparation process.

According to the weight of selected raw materials, the volatile oils were extracted from Pinellia ternate, Tangerine peel, Coix seed, Liquorice, Gastrodia elata, Rhizoma Arisaematis and Trichosanthes kirilowii respectively by supercritical fluid extraction or organic solvent extraction method. the volatile oils were mixed, and the conventional auxiliary materials were added. The pharmaceutical formulations such as soft capsules or granules were made according to the conventional preparation process.

According to the weight of the selected raw materials, the alkaloids and volatile oils were extracted from Pinellia ternata, Tangerine peel, Coix seed, Liquorice, Gastrodia elata, Rhizoma Arisaematis and Trichosanthes kirilowii respectively by supercritical fluid extraction or organic solvent extraction or steam distillation. The volatile oils were mixed with various alkaloids and some auxiliary materials after inclusion with betamethasin in proportion, and then mixed with various alkaloid dryers and proper accessories The method of preparing medicine is to make the pharmaceutically acceptable dosage forms such as tablet and granule.

### Example 6

The composition of the medicine for treating pulmonary obstructive fungal disease in the embodiment is as follows: alkaloids and volatile oils in 100 g of Pinellia ternata, alkaloids and volatile oils in 100 g of Tangerine peel, alkaloids and volatile oils in 60 g of fresh Liquorice, alkaloids and volatile oils in 150 g of fresh Coix seed, alkaloids and volatile oils in 100 g of fresh Gastrodia elata, alkaloids and volatile oils in 100 g of fresh Radix Stemonae, alkaloids and volatile oils in 120 g of Japanese ardisia, alkaloids and volatile oils in 100 g of fresh Lotus root.

The alkaloids were extracted from Pinellia ternata, Tangerine peel, Coix seed, Liquorice, Gastrodia elata, Radix Stemonae, Japanese ardisia and Lotus root joint by supercritical fluid extraction or membrane separation according to the weight of the selected raw materials. The alkaloids were mixed, added with conventional auxiliary materials, and the pharmaceutical acceptable formulations such as tablets or powder injection were made according to the conventional preparation process.

The volatile oils were extracted from Pinellia ternata, Tangerine peel, Coix seed, Liquorice, Gastrodia elata, Radix Stemonae, Japanese ardisia and Lotus root joint by supercritical fluid extraction or organic solvent extraction or steam distillation according to the weight of selected raw materials. Then, the volatile oil was mixed with the volatile oil, and the conventional preparation of drugs was used to make soft capsules or granules The dosage form of.

The alkaloids and volatile oils of the raw materials were extracted from Pinellia ternata, Tangerine peel, Coix seed, Liquorice, Gastrodia elata, Radix Stemonae, Japanese ardisia and Lotus root joint by supercritical fluid extraction or organic solvent extraction or steam distillation according to the weight of selected raw materials. The volatile oils were mixed with various alkaloids dry and proper auxiliary materials after inclusion with betacyclin, so as to The conventional method of preparing drugs can be used to make the pharmaceutically acceptable formulations such as tablets and granules.

### Example 7

The composition of the medicine for treating pulmonary mycosis in the case of pulmonary edema and pleural effusion in the embodiment is as follows: alkaloids and volatile oils in 120 g fresh Pinellia ternata, alkaloids and volatile oils in 80 g fresh Tangerine peel, alkaloids and volatile oils in 80 g fresh Liquorice, alkaloids and volatile oils in 200 g fresh Coix seed, alkaloids and volatile oils in 150 g fresh Gastrodia elata, alkaloids and volatile oils in 100 g fresh Japanese ardisia, alkaloids and volatile oils in 100 g fresh Rhizoma Arisaematis, alkaloids and volatile oils in 80 g fresh Aconitum carmichaeli and alkaloids and volatile oils in 100 g fresh Hovenia dulcis.

According to the weight of the selected raw materials, the alkaloids were extracted from Pinellia ternate, Tangerine peel, Coix seed, Liquorice, Gastrodia elata, Japanese ardisia, Rhizoma Arisaematis, Aconitum carmichaeli and Hovenia dulcis by supercritical fluid extraction or membrane separation method. The alkaloids were mixed and added with conventional excipients to prepare tablets or powder injections according to the conventional preparation process.

According to the weight of selected raw materials, supercritical fluid extraction or organic solvent extraction method was used to extract the volatile oils from Pinellia ternata, Tangerine peel, Coix seed, Liquorice, Gastrodia elata, Japanese ardisia, Rhizoma Arisaematis, Aconitum carmichaeli and Hovenia dulcis. Then the volatile oil was mixed and added with conventional excipients. According to the conventional preparation process, the pharmaceutically acceptable dosage forms such as soft capsules or granules were made.

According to the weight of the selected raw materials, the alkaloids and volatile oils were extracted from Pinellia ternata, Tangerine peel, Coix seed, Liquorice, Gastrodia elata, Japanese ardisia, Rhizoma Arisaematis, Aconitum carmichaeli and Hovenia dulcis by supercritical fluid extraction, organic solvent extraction or steam distillation, respectively. The volatile oils were mixed in proportion, and then mixed with various alkaloids and appropriate amount of auxiliary substances after inclusion with beta cyclodextrin The mixture is made into tablets, granules and other pharmaceutically acceptable dosage forms by the conventional method of drug preparation.

Of course, the above embodiments are only for the purpose of clearly illustrating the examples given, rather than limiting the embodiments. For those skilled in the art, other changes or changes in different forms can be made on the basis of the above description. It is unnecessary and impossible to enumerate all the embodiments here, and the obvious changes or variations thus extended are still within the protection scope of the present invention.

Inventiveness and Novelty of the Invention

The creative idea of the invention starts from the research of headache case by Zhang Yuansu, a famous doctor of Jin Dynasty, thus cracking the theory of Yin Yang and five elements of ancient Chinese traditional culture. For the details of the decoding content of the theory of Yin Yang and five elements, please refer to my other application 201410299118.9 “a broad-spectrum anti anaerobic drug composition”, which introduces in detail the relationship between the theory of Yin Yang and five elements and modern medicine. The theory of Yin Yang and five elements is the basis of all the social theories in ancient China and the theory of traditional Chinese medicine. There are different interpretations of the theory of Yin Yang and five elements by Chinese physicians in the past dynasties. The mainstream view is that the initial formation of the theory of Yin Yang is related to the sun and the moon, and extends from the characteristics of “facing the sun” and “backing the sun” to all things. Traditional Chinese medicine believes that man is an organic whole composed of yin and Yang, and the fundamental mechanism of the occurrence, development and change of diseases lies in the imbalance of yin and Yang. Therefore, the basic principle of treating diseases is to adjust Yin and Yang, supplement its deficiency, reduce its excess, and restore the balance and coordination of yin and Yang in the body. Three yin and three yang are the basic forms of yin-yang theory of traditional Chinese medicine, but the relationship between yin-yang theory and modern medicine has not been clear in the field of traditional Chinese medicine. The mainstream view of traditional Chinese medicine is that the theory of Yin-Yang and five elements is not related to the theory of modern Western Medicine.

In traditional Chinese medicine, the bacteria living in the “Yin Meridian” are called “Yin evil”, which can be divided into “damp evil” and “cold evil”. Combined with modern medical knowledge, I believe that “Yin Qi” in traditional Chinese medicine refers to carbon dioxide, “Yang Qi” refers to oxygen. In traditional Chinese medicine, “Yin Meridian” refers to vein, “Yang Meridian” refers to artery. “Dampness evil” in “Yin Meridian” refers to various bacteria (such as fungi, anaerobes, bacilli, viruses, etc.) that survive in vein or mucus and are tolerant to hypoxia, while “cold evil” refers to mold. And because mold is a fungus, its vitality is very strong, and its tolerance to hypoxia is the strongest among all bacteria. Just like the principle of fermentation, all kinds of bacteria coexist at the beginning, but only mold survives after fermentation failure. Therefore, in the race against time, all kinds of hypoxia resistant bacteria can survive in the vein. Only mold has the strongest vitality and destructive power. Canceration is the process of fungi from quantitative change to qualitative change. The clinical symptoms reflected in the quantitative change process are the symptoms of various chronic diseases. When the symptoms of cancer appear, only fungi are dominant in the body. Therefore, I think fungi are the main pathogenic factors of cancer, and fungi are also the main pathogenic factors of various chronic diseases.

Therefore, I come to the conclusion that “Hua Shi medicine, Qu Han medicine” has antifungal effect. Inspired by the characteristics of acidophilic and alkalophilic fungi, I concluded that alkaloids contained in plant APIs are antifungal chemical components. Inspired by the traditional Chinese medicine “aromatizing dampness, pungent dampness, warm cold”, I concluded that volatile oil contained in plant APIs is also antifungal chemical components.

According to the theory of five elements, ancient scholars believe that wood, fire, earth, gold and water are the five basic substances that make up the whole world. This theory later infiltrated into traditional Chinese medicine, and combined with Yin-yang theory to become the basis of traditional Chinese medicine.

The characteristics of the five elements are: Wood—Qu Zhi, fire—Yan Shang, earth—farming, gold—Cong Ge, water—Run Xia. It is generally believed in the field of traditional Chinese medicine that although the characteristics of the five elements come from the specific observation of wood, fire, earth, metal and water, they are the result of the ancients' abstract description. They have gone beyond their own specific characteristics and have a broader rational meaning. Moreover, the traditional Chinese medicine circles have not been able to explain the characteristics of the five elements, especially the analysis of “farming” is far fetched.

Inspired by the word “Cang Jie”, I believe that “gold” is the anatomical figure of the lung, and “Wood” represents the whole body distribution map of twelve meridians, that is, the distribution map of blood vessels in the whole body, and the blood circulation path from foot to head and hand to head. “Earth” is the anatomical figure of liver, “water” is the anatomical figure of two kidneys and urinary system, “fire” is the anatomical figure of heart.

In traditional Chinese medicine, “mutual generation” refers to the fact that one of the five elements has the function of promoting, encouraging and supporting another. The law and order of the five elements are: wood makes fire, fire makes earth, earth makes gold, gold makes water and water makes wood. The so-called “mutual restraint” refers to the inhibition, restraint and weakening of one thing to another. The law and order of the five elements are: metal conquers wood, wood conquers earth, earth conquers water, water conquers fire and fire conquers gold. The application of the theory of five elements in traditional Chinese medicine is mainly to analyze and summarize the internal relations among the physiological functions of Jin Ke Mu, Mu Ke Tu, Tu Ke Shui, Shui Ke Huo and Huo Ke Jin by using the theory of five elements. In traditional Chinese medicine circles, it is believed that the pathological changes of one organ can affect other organs through different ways, and this organ itself may also be affected by the pathological changes of other organs. However, it has not been clear how they affect each other, nor has it been clear about the pathological mechanism of the five elements restraining each other.

My research on “wood-Mu” and “Earth-Tu” shows that “wood” does not represent the liver, but represents the whole body distribution map of twelve meridians, that is, the blood circulation path from foot to head and hand to head. In my opinion, “Earth” does not represent the spleen, but refers to the liver, “Earth” is the pictograph of the liver.

The relationship between the five elements indicates the process of pathological changes caused by pathogenic bacteria. Therefore, I think Jin ke Mu means: after the bacteria infect the lungs through the respiratory tract, they enter the blood circulation system of the whole body and stay in the capillary ends of hands, feet and heads. Especially in the upper respiratory tract, bacteria are easy to enter the brain capillaries. Fungi propagate continuously in the brain capillaries, blocking the veins and arteries of the brain. First, the clinical symptoms appear in the eyes, and then insomnia, mental disorders, hypertension and other symptoms.

Mu ke Tu means: bacteria in the blood stay at the end of capillaries of hands, feet and heads, continue to propagate and transfer, and stay in the liver to propagate, causing substantial pathological changes in the liver. Because the blood vessels of the liver are very rich, especially the hepatic portal vein group is easy to retain bacteria.

Tu ke Shui means: after invading the liver, the next pathogen causing pathological changes is the kidney. Because the blood vessels of the kidney are also very rich, normal people have 1000 ml blood flow through both kidneys every minute, glomerular capillaries are easy to retain bacteria, causing renal artery stenosis, mainly manifested as hypertension, renal vein thrombosis can appear a lot of proteinuria.

Shui ke Huo means: after the substantial pathological changes in the kidney, it is the turn for the heart to have substantial pathological changes.

Huo ke Jin: when there are pathological changes in the heart, the last thing is that there are substantial lesions in the lungs. This is because the lung is an open organ, with the most abundant oxygen content inside. Only when the number of fungi reaches a certain level and many blood vessels are blocked, can substantial lesions appear in the lung. At this time, the patient's systemic symptoms are obvious, which also indicates that the patient often dies of respiratory failure in the end.

Modern studies have found that the spores of fungi propagate by budding and form hyphae, which interweave into clusters. When the germinated spores grow to a certain size, they are separated from the mother. I think fungi are the same as human beings. When children grow up, they have to go out separately. Therefore, the fungi that fall off from the mother look for the suitable place for foot drop with the blood circulation everywhere, and the acupoints on the twelve meridians are the natural place for retention and reproduction.

Therefore, I think: the process of fungal pathological transfer is affected by non external factors, and the process of pathological transfer is: respiratory tract infection (entering from artery)→systemic blood circulation (end of capillaries in head, hand and foot)→hepatic vein and artery→renal vein and artery→cardiac vein and artery→pulmonary vein and artery.

Of course, trauma, surgery, transplantation and other external factors will lead to changes in the process of fungal pathological metastasis. Moreover, fungi are easy to stay and reproduce in the injured part, because the blood circulation of the injured part is blocked.

The medicine provided by the invention is innovated on the basis of Er Chen Tang. The medicine provided by the invention is made by mixing the alkaloids and/or volatile oils contained in Pinellia ternata, Tangerine feel, Liquorice, Coix seed, Gastrodia elata and other plant medicines on the basis of Pinellia ternata, tangerine peel, Liquorice, Coix seed, Gastrodia elata.

The inventiveness of the medicine provided by the invention is embodied in that the raw material medicine is composed of Pinellia alkaloids, Tangerine peel alkaloids, Liquorice alkaloids, Coix seed alkaloids, Gastrodia elata alkaloids, etc., instead of pinellia, Tangerine peel, Liquorice, Coix seed, Gastrodia elata and other natural herbs. The alkaloids extracted from Pinellia ternate, Tangerine, Liquorice, Coix seed and Gastrodia elata were mixed in proportion, and the conventional excipients were added to make the clinically acceptable dosage form according to the conventional preparation process.

Compared with the existing chemical drugs which are made from single plant alkaloids, the drug provided by the invention can reduce the content of single alkaloid in the drug, reduce the toxic reaction caused by excessive dosage of single alkaloid, and improve the drug resistance. For example, morphine obtained from opium has a strong analgesic effect, because it is a single alkaloid, in order to achieve the curative effect, its dosage is too large and prone to toxic and side effects. Moreover, the medicine provided by the invention can more effectively target the vascular tissues in different parts due to the synergistic effect of multiple alkaloids, and has more drug resistance.

Compared with the traditional Chinese medicine made by the modern pharmaceutical method, the medicine provided by the invention also has significant progress. Because traditional Chinese medicine is not necessarily all herbal medicine, but also animal and mineral, the most important feature of modern traditional Chinese medicine is that it is unable to know all the ingredients, but only the main ingredients, that is, the chemical ingredients that can cure diseases. The chemical ingredients of the medicine provided by the invention are only various alkaloids and volatile oils, and the chemical ingredients are clear.

Moreover, the pharmacological mechanism of the existing Chinese patent medicine is not clear, and the pharmaceutical pharmacological mechanism provided by the invention is clear. The pharmacological mechanism is that the alkaloids of each API have antifungal effect, and the volatile oil of each API has antifungal effect. Moreover, different alkaloids and volatile oils have their own characteristics, which can target different blood vessels, tissues and cells. Therefore, the combination of alkaloids and/or volatile oil can play a team role. The effectiveness of alkaloids and volatile oil can be significantly improved by cooperation and division of labor.

### Technical Effect

The traditional Er Chen Tang has been clinically proved to be safe and effective. The applicant treated an elderly patient with pulmonary aspergillosis by traditional Chinese medicine decoction with raw materials such as Pinellia ternata, Tangerine peel, Liquorice, Coix seed and Gastrodia elata. After 7 days, the symptoms were significantly improved. The patient was chronic bronchitis, long-term cough, sputum with blood, common anti-inflammatory treatment failed repeatedly, suspected of fungal infection, sputum smear found Aspergillus hyphae. The medicine provided by the invention is based on the raw material medicine of the traditional classical prescription Er Chen Tang, and extracts the volatile oils and alkaloids contained in the plant medicine Pinellia ternate, Tangerine peel, Liquorice, Coix seed, Gastrodia elata to prepare the medicine or health care product for treating pulmonary fungal disease. Because the chemical composition of the medicine or health care product provided by the invention is clear and the pharmacological action is clear, the effectiveness is obvious compared with the traditional pharmaceutical method. Because the applicant has no conditions to extract alkaloids and volatile oils from the API for test, however, based on the contents recorded in the description and the common technical knowledge in the art, those skilled in the art can implement the technical scheme of the invention, solve the technical problems and produce the expected technical effect.

1. A medicine for treating pulmonary mycosis, characterized in that the raw materials for the medicine include alkaloids and/or volatile oils extracted from Pinellia ternata, alkaloids and/or volatile oils extracted from Tangerine peel, alkaloids and/or volatile oils extracted from Liquorice, alkaloids and/or volatile oils extracted from Coix seed, alkaloids and/or volatile oils extracted from Gastrodia elata; the methods for preparing the medicine include: taking Pinellia ternata alkaloids, Tangerine peel alkaloids, Coix seed alkaloids, Liquorice alkaloids, and Gastrodia elata alkaloids, mixing them in a certain proportion, adding various conventional excipients required for preparing different dosage forms to them, and preparing pharmaceutically acceptable dosage forms; taking Pinellia ternate volatile oils, fresh Tangerine peel volatile oils, Liquorice volatile oils, Coix seed volatile oils, and Gastrodia elata volatile oils, mixing them in a certain proportion, adding various conventional excipients required for preparing different dosage forms to them, and preparing pharmaceutically acceptable dosage forms; taking Pinellia ternate alkaloids and volatile oils, Tangerine peel alkaloids and volatile oils, Liquorice alkaloids and volatile oils, Coix seed alkaloids and volatile oils, Gastrodia elata alkaloids and volatile oils, mixing the volatile oils in a proportion, including beta-cyclodextrin in them, mixing them with various dry alkaloid substances and suitable amounts of excipients, and preparing pharmaceutically acceptable dosage forms using conventional methods for preparing medicines.

2. The medicine according to claim 1, characterized in that the raw materials for the medicine can be further selected from: alkaloids and/or volatile oils extracted from Fritillaria cirrhosa, alkaloids and/or volatile oils extracted from Trichosanthes kirilowii, alkaloids and/or volatile oils extracted from Rhizoma arisaematis, alkaloids and/or volatile oils extracted from Sinapis alba, alkaloids and/or volatile oils extracted from Gleditsia sinensis, alkaloids and/or volatile oils extracted from Radix stemona, alkaloids and/or volatile oils extracted from Japanese ardisia, alkaloids and/or volatile oils extracted from Artichoke, alkaloids and/or volatile oils extracted from Bletilla striata, alkaloids and/or volatile oils extracted from Lotus root, alkaloids and/or volatile oils extracted from Aconitum carmichaeli, alkaloids and/or volatile oils extracted from Hovenia dulcis.

3. The medicine according to claim 1, characterized in that the weight parts of the alkaloids and/or volatile oils of the raw materials for the medicine are: 0.001-50 weight parts of the alkaloids and/or volatile oils extracted from Pinellia ternata, 0.001-50 weight parts of the alkaloids and/or volatile oils extracted from Tangerine peel, 0.001-50 weight parts of the alkaloids and/or volatile oils extracted from Coix seed, 0.001-50 weight parts of the alkaloids and/or volatile oils extracted from Liquorice, 0.001-50 weight parts of the alkaloids and/or volatile oils extracted from Gastrodia elata, 0-50 weight parts of the alkaloids and/or volatile oils extracted from Fritillaria cirrhosa, 0-50 weight parts of the alkaloids and/or volatile oils extracted from Trichosanthes kirilowii, 0-50 weight parts of the alkaloids and/or volatile oils extracted from Rhizoma arisaematis, 0-50 weight parts of the alkaloids and/or volatile oils extracted from Sinapis alba, 0-50 weight parts of the alkaloids and/or volatile oils extracted from Gleditsia sinensis, 0-50 weight parts of the alkaloids and/or volatile oils extracted from Radix stemona, 0-50 weight parts of the alkaloids and/or volatile oils extracted from Japanese ardisia, 0-50 weight parts of the alkaloids and/or volatile oils extracted from Artichoke, 0-50 weight parts of the alkaloids and/or volatile oils extracted from Bletilla striata, 0-50 weight parts of the alkaloids and/or volatile oils extracted from Lotus root, 0-50 weight parts of the alkaloids and/or volatile oils extracted from Aconitum carmichaeli, 0-50 weight parts of the alkaloids and/or volatile oils extracted from Hovenia dulcis.

4. The medicine according to claim 1, characterized in that the methods for preparing the medicine include: according to the weight parts of the selected raw materials, extracting alkaloids from Pinellia ternata, Tangerine peel, Liquorice, Coix seed, Gastrodia elata or Fritillaria cirrhosa or Trichosanthes kirilowii or Rhizoma arisaematis or Sinapis alba or Gleditsia sinensis or Radix stemona or Japanese ardisia or Artichoke or Bletilla striata or Lotus root or Aconitum carmichaeli or Hovenia dulcis, respectively, using modern extraction technology, such as, but not limited to, cold immersion, percolation, supercritical fluid extraction, membrane separation, ultrasonic extraction, Soxhlet extraction method; mixing the respective alkaloids, and adding conventional excipients to them; preparing pharmaceutically acceptable dosage forms, such as, but not limited to, tablets, powder injections and capsules, according to conventional processes for preparing medicines.

5. The medicine according to claim 1, characterized in that the methods for preparing the medicine include: according to the weight parts of the selected raw materials, extracting volatile oils from Pinellia ternata, Tangerine peel, Liquorice, Coix seed, Gastrodia elata or Fritillaria cirrhosa or Trichosanthes kirilowii or Rhizoma arisaematis or Sinapis alba or Gleditsia sinensis or Radix stemona or Japanese ardisia or Artichoke or Bletilla striata or Lotus root or Aconitum carmichaeli or Hovenia dulcis, respectively, using modern extraction technology, such as, but not limited to, supercritical fluid extraction, organic solvent extraction, steam distillation and cold pressing method; mixing various volatile oils in a proportion and adding various conventional excipients required for preparing different dosage forms to them; preparing pharmaceutically acceptable dosage forms, such as, but not limited to, tablets, soft capsules, topical liniment, and granules, according to conventional processes for preparing medicines.

6. The medicine according to claim 1, characterized in that the methods for preparing the medicine include: according to the weight parts of the selected raw materials, extracting alkaloids and volatile oils from Pinellia ternata, Tangerine peel, Liquorice, Coix seed, Gastrodia elata or Fritillaria cirrhosa or Trichosanthes kirilowii or Rhizoma arisaematis or Sinapis alba or Gleditsia sinensis or Radix stemona or Japanese ardisia or Artichoke or Bletilla striata or Lotus root or Aconitum carmichaeli or Hovenia dulcis, respectively, using modern extraction technology, such as, but not limited to, supercritical fluid extraction, membrane separation, organic solvent extraction, steam distillation, cold pressing method; mixing the various volatile oils in a proportion, including beta-cyclodextrin in them, mixing them with various dry alkaloid substances and suitable amounts of excipients, adding various conventional excipients required for preparing different dosage forms to them; preparing pharmaceutically acceptable dosage forms, such as, but not limited to, tablets and granules, according to conventional processes for preparing medicines.

7. The medicine according to claim 1, characterized in that the scope of application of the medicine includes application in medical medicine, health food, health products, animal medicine, and animal feed.
